(19) United States
(12) Patent Application Publication
(10) Pub. No.: US 2026/0144804 A1
(43) Pub. Date: May 28, 2026

(54) USE OF HYOCHOLIC ACID (HCA) SPECIES IN PREPARATION OF MEDICAMENT FOR TREATMENT OF IRRITABLE BOWEL SYNDROME WITH DIARRHEA

(71) Applicant: Centre for Chinese Herbal Medicine Drug Development Limited, Shatin (HK)

(72) Inventors: Zhaoxiang BIAN, Shatin (HK); Lixiang ZHAI, Shatin (HK); Xavier Hoi Leong WONG, Shatin (HK); Ziwan NING, Shatin (HK); Shujun XU, Shatin (HK); Junfang LYU, Shatin (HK); Gengyu BAO, Shatin (HK)

(21) Appl. No.: 19/176,024

(22) Filed: Apr. 10, 2025

(30) Foreign Application Priority Data
Nov. 25, 2024 (CN) 202411698580.6

(51) Int. Cl. A61K 31/575; A61K 35/413; A61P 1/00; A61P 1/12

(52) U.S. Cl. CPC A61K 31/575; A61K 35/413; A61P 1/00; A61P 1/12

(57) ABSTRACT

The present invention discloses use of hyocholic acid (HCA)-based bile acid species in the preparation of a medicament for the treatment of irritable bowel syndrome (IBS). It was discovered in the present invention that the content of HCA species in the feces of IBS-D patients treated with traditional Chinese medicine was significantly increased. Moreover, HCA species were significantly reduced in fecal samples of IBS-D patients compared with healthy controls. Further research revealed that HDCA and natural traditional Chinese medicine PULVIS FELLIS SUIS can reduce the peripheral serotonin level by inhibiting the activity of tryptophan hydroxylase 1, and significantly improve the symptoms of diarrhea and abdominal pain in TNBS-induced mice, indicating an application value of HCA species in treating IBS-D.

### CROSS-REFERENCE TO RELATED APPLICATION

This application claims priority to Chinese Patent Application No. 202411698580.6, filed on Nov. 25, 2024, which is hereby incorporated by reference in its entirety.

### TECHNICAL FIELD

The present invention belongs to the field of biomedicine, and specifically relates to use of hyocholic acid (HCA) species in the preparation of medicaments for the treatment of irritable bowel syndrome with diarrhea (IBS-D).

### BACKGROUND

Irritable Bowel Syndrome with Diarrhea (IBS-D) is a common gastrointestinal disorder characterized by frequent diarrhea, abdominal pain, and bloating. Current treatment for IBS-D mainly relieves uncomfortable symptoms such as diarrhea and abdominal pain, but cannot effectively cure IBS-D, and recurrent diarrhea and abdominal discomfort significantly impair patients' quality of life and cause emotional stress. Serotonin (also known as 5-HT) plays an important role in regulating gastrointestinal motility and visceral sensitivity. In IBS-D patients, abnormalities in biosynthesis and reuptake of serotonin, as well as excessive proliferation of enterochromaffin cells, result in increased release of serotonin into intestinal tissue. This accelerates gastrointestinal motility, enhances fluid secretion in the intestinal lumen and visceral hypersensitivity, and exacerbates symptoms such as diarrhea and abdominal pain. Recent studies have demonstrated a significant causal relationship between gut microbial metabolites and the regulation of peripheral serotonin. These gut bacteria modulate the proliferation of enterochromaffin cells and in turn influence gut motility and visceral sensitivity by producing specific gut-microbial metabolites such as short-chain fatty acids, deoxycholic acid, and aromatic trace amines.

### SUMMARY

The present inventors have discovered that HCA species, including hyocholic acid (HCA), hyodeoxycholic acid (HDCA), glycohyocholic acid (GHCA), glycohyodeoxycholic acid (GHDCA), taurohyocholic acid (THCA), and taurohyodeoxycholic acid (THDCA), directly bind to tryptophan hydroxylase 1 (TPH1), the rate-limiting enzyme for peripheral serotonin biosynthesis, thereby specifically inhibiting serotonin overproduction in IBS-D patients. This mechanism is essential for alleviating diarrhea and abdominal pain, as demonstrated in TNBS-induced IBS mice treated with HDCA or PULVIS FELLIS SUIS.

The invention thus provides HCA species and PULVIS FELLIS SUIS for use in treating IBS-D via TPH1-serotonin pathway modulation, enabling the preparation of pharmaceutical compositions with optimized dosage forms (e.g., oral tablets, injectables) and dosing regimens (0.1-50.0 mg/kg once daily).

In some embodiments, the HCA species comprises one or more selected from a group consisting of hyocholic acid (HCA), hyodeoxycholic acid (HDCA), glycohyocholic acid (GHCA), glycohyodeoxycholic acid (GHDCA), taurohyocholic acid (THCA), and taurohyodeoxycholic acid (THDCA).

In some embodiments, the HCA species include HCA and/or HDCA and/or GHCA and/or GHDCA and/or THCA and/or THDCA.

According to a specific embodiment of the present invention, the crude drug comprising HCA species comprises PULVIS FELLIS SUIS.

According to a specific embodiment of the present invention, the HCA species or the crude drug comprising HCA species can inhibit the activity of TPH1 or the production of serotonin.

According to a specific embodiment of the present invention, the pharmaceutical composition or pharmaceutical formulation comprises a pharmaceutically acceptable carrier and/or auxiliary material.

In the present invention, the pharmaceutically acceptable carrier includes, but is not limited to, a diluent, a binder, a surfactant, a humectant, an adsorbent carrier, a lubricant, a filler, or a disintegrant.

In some embodiments, the dosage form of the pharmaceutical formulation includes dosage forms for injection, dosage forms for respiratory tract administration, dosage forms for administration via a cavity or tract, dosage forms for mucosal administration, or dosage forms for administration to or via skin.

In some embodiments, the dosage form of the pharmaceutical formulation includes a tablet, a capsule, granules, pills, dripping pills, syrup, powder, pulvis, a suppository, drops, an emulsion, an injection, a solution, or a suspension.

In some embodiments, the administrative dose of the HCA species is 0.1-50.0 mg/kg, and the frequency of administration is once a day.

In some embodiments, the administrative dose of the HCA species is 11.1-16.6 mg/kg, and the frequency of administration is once a day.

In some embodiments, the HCA species or the crude drug comprising HCA species can improve the diarrhea and abdominal pain phenotypes. According to a specific embodiment of the present invention, the diarrhea and abdominal pain phenotypes involve the gastrointestinal transit, defecation frequency, or fecal moisture content.

The present invention also provides a method for diagnosing whether an individual suffers from IBS-D, comprising: providing a biological sample from an individual; detecting a content of HCA species in the biological sample.

In some embodiments, the method for diagnosing whether an individual suffers from IBS-D further comprising comparing the content of the HCA species in the biological sample with that of a biological sample from a healthy individual to diagnose whether the individual suffers from IBS-D.

The present invention also provides a pharmaceutical composition for treating IBS-D, comprising HCA species and/or a crude drug comprising HCA species.

In some embodiments, the HCA species comprises one or more selected from a group consisting of hyocholic acid (HCA), hyodeoxycholic acid (HDCA), glycohyocholic acid (GHCA), glycohyodeoxycholic acid (GHDCA), taurohyocholic acid (THCA), and taurohyodeoxycholic acid (THDCA).

According to a specific embodiment of the present invention, the HCA species include HCA and/or HDCA and/or GHCA and/or GHDCA and/or THCA and/or THDCA.

According to a specific embodiment of the present invention, the crude drug comprising HCA species is PULVIS FELLIS SUIS.

In some embodiments, a dosage form of the pharmaceutical composition includes dosage forms for injection, dosage forms for respiratory tract administration, dosage forms for administration via a cavity or tract, dosage forms for mucosal administration, and dosage forms for administration to or via skin.

The present invention also provides a method for preventing or treating Irritable Bowel Syndrome with Diarrhea (IBS-D), comprising administering a therapeutically effective amount of hyocholic acid based bile acid species (HCA species) and/or a crude drug comprising HCA species to a subject in need thereof.

The term “subject” refers to an animal, including, but not limited to, a primate (e.g., human), cow, sheep, goat, horse, dog, cat, rabbit, rat, or mouse.

In some embodiments, the HCA species comprises one or more selected from a group consisting of hyocholic acid (HCA), hyodeoxycholic acid (HDCA), glycohyocholic acid (GHCA), glycohyodeoxycholic acid (GHDCA), taurohyocholic acid (THCA), and taurohyodeoxycholic acid (THDCA).

According to a specific embodiment of the present invention, the HCA species include HCA and/or HDCA and/or GHCA and/or GHDCA and/or THCA and/or THDCA.

According to a specific embodiment of the present invention, the crude drug comprising HCA species is PULVIS FELLIS SUIS.

In some embodiments, the HCA species or the crude drug comprising HCA species is capable of inhibiting TPH1 activity or production of peripheral serotonin.

In some embodiments, the HCA species and/or crude drug comprising HCA species is administered by injection, by administration via the respiratory tract, by administration via a cavity or tract, by mucosal administration, or by cutaneous administration.

In some embodiments, the HCA species is administered with a dose of 0.1-50.0 mg/kg, and is administered at a frequency of once a day.

In some embodiments, the HCA species is administered with a dose of 11.1-16.6 mg/kg, and is administered at a frequency of once a day.

In some embodiments, the HCA species or the crude drug comprising HCA species is capable of improving the diarrhea and abdominal pain phenotypes.

In the present invention, the administration route of the HCA species includes, but is not limited to, intravenous injection, subcutaneous injection, intramuscular injection, transdermal administration, topical administration, implantation, or sustained release administration.

The present invention also provides use of HCA species or a crude drug comprising HCA species in the preparation of a tryptophan hydroxylase 1 inhibitor (TPH1).

According to a specific embodiment of the present invention, the HCA species include HCA and/or HDCA and/or GHCA and/or GHDCA and/or THCA and/or THDCA.

In some embodiments, the HCA species or the crude drug comprising HCA species inhibits serotonin biosynthesis by inhibiting TPH1 activity.

The present invention also provides a method for inhibiting TPH1 activity in a subject with IBS-D, comprising: administering HCA species or a crude drug comprising HCA species to the subject.

### Beneficial Effects

In the invention, for the first time, HCA species were found to have significant association with peripheral serotonin in IBS-D patients and be negatively correlated with the diarrhea and abdominal pain phenotypes and the peripheral serotonin level in the patients. By evaluation of the effects of HCA species, in particular HDCA and the natural traditional Chinese medicine PULVIS FELLIS SUIS, on the diarrhea and abdominal pain phenotypes and the peripheral serotonin level in TNBS-induced IBS mice, it was found that HDCA and the natural traditional Chinese medicine PULVIS FELLIS SUIS significantly improved the diarrhea and abdominal pain symptoms in the TNBS-induced mice. Our data indicate that HDCA can be used to treat IBS-D by inhibiting serotonin signaling.

The present inventors have discovered that HCA species, including HCA, HDCA, GHCA, GHDCA, THCA, and THDCA, were significantly reduced in the feces of IBS-D patients, and were negatively correlated with the diarrhea and abdominal pain symptoms and the peripheral serotonin level in IBS-D patients, suggesting that HCA species may be involved in the regulation of the peripheral serotonin level and may alleviate the diarrhea and abdominal pain symptoms associated with IBS-D.

The content of HCA species is relatively low in the human body, constituting 2.43% of the total bile acids. In contrast, the total content of HCA species in pigs accounts for 76.34% of the total bile acids. Therefore, supplementing exogenous HCA species to improve the diarrhea and abdominal pain symptoms of IBS-D appears to be a feasible strategy. The Chinese medicine PULVIS FELLIS SUIS is dried bile products of Sus scrofa domestica Brisson, and contains a high level of HCA species, suggesting that it can be used as a natural medicine to regulate the peripheral serotonin level and improve the diarrhea and abdominal pain symptoms in IBS-D patients.

Our results show that HDCA and PULVIS FELLIS SUIS can significantly improve the diarrhea and abdominal pain phenotypes and modulate the abnormal peripheral serotonin level in TNBS-induced mice. In vitro experiments demonstrated that HCA species suppress the level of serotonin secreted by the QGP-1 cell line. Moreover, the inhibition of tryptophan hydroxylase 1 (TPH1) activity by HCA species is targeted therapeutic strategy specific to IBS-D, where dysregulated serotonin directly contributes to gastrointestinal symptoms. This distinguishes HCA species from broad-spectrum TPH1 inhibitors.

### BRIEF DESCRIPTION OF THE DRAWINGS

FIG. 1 shows that HCA species were negatively correlated with the peripheral serotonin level and gastrointestinal symptoms in IBS-D patients. FIG. 1 a-g show that HCA species, including HCA and HDCA, were significantly reduced in the feces of IBS-D patients (n=141) compared to healthy controls (n=57). FIG. 1 h-j show that according to a non-parametric Spearman correlation analysis, the level of total fecal HCA species in healthy controls and IBS-D patients (black is healthy controls, red is IBS-D patients) was negatively correlated with the peripheral serotonin level, defecation frequency, and abdominal pain index.

FIG. 2 shows that HCA species were regulated by gut microbiota and were dominant in total bile acids from pigs. FIG. 2 a-f show the level of total HCA species, including HCA and HDCA, in feces and serum samples of mice raised under conventional feeding conditions and of germ-free mice; FIG. 2 g-h show the proportion of total HCA species in PULVIS FELLIS SUIS and in total bile acids of PULVIS FELLIS SUIS.

FIG. 3 shows that PULVIS FELLIS SUIS and HDCA improved the diarrhea and abdominal pain phenotypes in a TNBS-induced IBS mice model. FIG. 3 a-c show the effects of PULVIS FELLIS SUIS and HDCA on the diarrhea-like phenotypes in the TNBS-induced IBS mice model, including the GI transit time, fecal water content and defecation frequency; FIG. 3 d-f show the effects of HDCA and PULVIS FELLIS SUIS on the abdominal pain-like phenotypes in the TNBS-induced IBS mice model, including the area under the electromyographic curve (AUC) after 30/60/90 μL target water volumes were given.

FIG. 4 illustrates that HCA species inhibited the serotonin level both in vitro and in vivo. FIG. 4 a-f show the effects of HCA species, including HCA, HDCA, GHCA, GHDCA, THCA, and THDCA, on the serotonin level produced by a QGP-1 cell line. FIG. 4 g shows the effects of HDCA and PULVIS FELLIS SUIS on the serotonin level in an ileum sample from the TNBS-induced IBS mouse model.

FIG. 5 shows that HDCA suppresses the biosynthesis of serotonin by inhibiting the TPH1 activity. FIG. 5 a shows the inhibitory effect of HDCA on human TPH1. FIG. 5 b shows the interaction between HDCA and recombinant human TPH1.

### DESCRIPTION OF THE EMBODIMENTS

Before further describing the specific embodiments of the present invention, it should be understood that the scope of protection of the present invention is not limited to the specific embodiments described later; and it should be further understood that the terms used in the examples of the present invention are intended to describe the specific embodiments and not to limit the scope of protection of the present invention.

When numerical ranges are given in the embodiments, it should be understood that, unless otherwise specified, the two endpoints of each numerical range and any numerical values between the two endpoints can be implemented. Unless otherwise defined, all technical and scientific terms used herein have the same meanings as commonly understood by those skilled in the art. In addition to the specific methods, apparatus, and materials used in the examples, any methods, apparatus or materials in the prior art that are similar to or equivalent to those described in the examples of the present invention may also be used to carry out the invention based on the knowledge of those skilled in the art and the description of the present invention.

Unless otherwise stated, the experimental methods, testing methods, and preparation methods not disclosed in the present invention are conventional techniques in the art.

Through preliminary research, the present inventors discovered that the level of fecal HCA species in IBS-D patients was significantly decreased compared to that in healthy controls, and was negatively correlated with the diarrhea and abdominal pain symptoms and the serotonin level in IBS-D patients, suggesting that HCA species may improve the diarrhea and abdominal pain symptoms of IBS-D by regulating the peripheral serotonin level. PULVIS FELLIS SUIS, as a natural traditional Chinese medicine, contains a relatively high content of HCA species, suggesting that it may act as an exogenous metabolite to regulate the peripheral serotonin level and improve the diarrhea and abdominal pain symptoms of IBS-D. Further studies showed that HDCA and PULVIS FELLIS SUIS can significantly improve the diarrhea and abdominal pain phenotypes and inhibit the serotonin level in a TNBS-induced IBS animal model. In in vitro and in vivo experiments, HCA species were shown to inhibit the synthesis and reuptake of serotonin in the QGP-1 cell line. The present data indicate a new use of HCA species for the treatment of IBS-D by inhibiting serotonin signaling. In vitro experiments revealed that HDCA can inhibit TPH1 activity and directly binds to TPH1, indicating that HDCA is an effective TPH1 inhibitor.

### Example 1

### Materials and Methods

### 1. Reagents

2,4,6-trinitrobenzene sulfonic acid (TNBS, Sigma, Cat. p2297), Ethanol (Honeywell, Cat. 32221-2.5L), Isoflurane (Piramal, Lot. N0791K11), Hyocholic acid (HCA, Sigma, Cat.700159P, CAS.547-75-1), Hyodeoxycholic acid (HDCA, MedChemExpress, Cat.HY-N0169), Glycohyocholic acid (GHCA, Cayman Chemical, Cat.22670, CAS.32747-08-3), Glycohyodeoxycholic acid (GHDCA, Cayman Chemical, Cat.22643, CAS.13042-33-6), Taurohyocholic acid (THCA, Cayman Chemical, Cat.22669, CAS.117997-17-8), Taurohyodeoxycholic acid (THDCA, Cayman Chemical, Cat.21956, CAS.38411-85-7), PULVIS FELLIS SUIS (Solarbio, Cat.LA1090), Serotonin (Sigma, Cat.14927, CAS.50-67-9), L-Tryptophan-d₅ (MedChemExpress, Cat.HY-N0623S, CAS.62595-11-3), Cholic acid-d4 (MedChemExpress, Cat.HY-N0324S, CAS.116380-66-6), Deoxycholic acid-d4 (MedChemExpress, Cat.HY-N0593S, CAS.112076-61-6), Carmine (Macklin, Cat.C805213, CAS.1390-65-4), Sodium carboxymethyl cellulose (Chem Scene, Cat.CS-0015715, CAS.9004-32-4), RPMI Medium 1640 powder (Gibco, Cat.31800-022), Sodium bicarbonate powder (Acros Organics, Cat.AC123360010, CAS.144-55-8), Fetal bovine serum (FBS, Gibco. Cat. #10099), Series S Sensor Chip CM5 (Cytiva, Cat. 29104988), Acetate 4.0 (Cytiva, Cat.BR100349), Amine Coupling kit (Cytiva, Cat.BR100050), PBS 10× (Cytiva, Cat.BR100672), TPH1 Inhibitor Screening Assay Kit (BPS bioscience, Cat.72056), TPH1 (His-tag (Human) Recombinant (BPS bioscience, Cat.71192)).

### 2. Methods

### 2.1 Human Studies

IBS-D patients (n=345) meeting Rome IV diagnostic criteria and healthy subjects as controls (n=91) were recruited, and written informed consent was obtained from each participant prior to specimen collection. Biological samples, including serum, urine, and feces from all participants, were collected and immediately placed on dry ice, and were frozen at −80° C. in the laboratory until use. This human study was approved by the Research Ethics Committee of Hong Kong Baptist University (HASC/15-16/0300 and HASC/16-17/0027).

### 2.2 Animal Experiments

Male C57BL/6J mice (6-8 weeks old) were purchased from Jicui Yaokang Co., Ltd. (Jiangsu, China). The mice were housed in chambers with a 12-hour light/dark cycle at a constant temperature (22±2° C.) and humidity (40%-70%), allowed free access to a standard rodent diet and water.

### 2.3 Post-Infection Irritable Bowel Syndrome (PI-IBS) Model

C57BL/6J mice were fasted overnight and provided with drinking water containing 5% glucose. After the mice were weighed and anesthetized, a 100 μL TNBS solution containing 30% ether was slowly injected into the colonic lumen using a 1 mL syringe and 3.5F catheter. Subsequently, the catheter was gently removed from the colon of the mice, and the mice were maintained in a head-down vertical position for 5 min. The control group received physiological saline as a control. Mice were weighed on days 1-5, 14, and 21 following TNBS administration.

### HDCA and PULVIS FELLIS SUIS Treatment

PI-IBS mice were randomly divided into four groups 19 days after modeling: 1) Model group: Mice (n=6) were administered with 300 μL vehicle solution containing 0.5% sodium carboxymethylcellulose; 2) Low-dose HDCA group: Mice (n=6) were administered with 300 μL sodium carboxymethylcellulose solution containing 100 mg/kg HDCA; 3) High-dose HDCA group: Mice (n=6) were administered with 300 μL sodium carboxymethylcellulose solution containing 150 mg/kg HDCA; 4) PULVIS FELLIS SUIS group: Mice (n=6) were administered with 300 μL sodium carboxymethylcellulose solution containing 100 mg/kg PULVIS FELLIS SUIS. A control group was also set up: healthy mice (n=6) were given 300 μL control vehicle solution containing 0.5% sodium carboxymethylcellulose.

The GI transit time, fecal moisture content, and defecation frequency were measured on day 12 after administration with 2,4,6-trinitrobenzene sulfonic acid solution and on days 7 and 14 after treatment, to determine whether administration with HDCA and PULVIS FELLIS SUIS affected the diarrhea and abdominal pain-like symptoms in the IBS mice model.

### GI Transit Time

The mice were placed individually in sterile cages and given 6% carmine solution (dissolved in a 0.5% methylcellulose solution, w/v) by gavage and observed. At 9:00 a.m. before the experiment started, each mouse without fasting was given 300 μL carmine solution by gavage, and the time from gavage to initial appearance of carmine in the feces was recorded as the GI transit time for that mouse.

### Evaluation of Defecation Frequency and Fecal Moisture Content

The mice were placed individually in sterile cages, and the cumulative number of fecal pellets of each mouse within 120 min was recorded as corresponding defecation numbers. The fresh fecal pellets from each mouse were collected immediately after defecation. The wet weight and dry weight of each fecal pellet before and after drying in an oven (at 105° C. for 24 h) was recorded. The fecal moisture content (%) was calculated as follows:

$$Fecal water content ( \% ) = ( {W}_{wet} - {W}_{dry} ) / {W}_{wet} \times 100.$$

### Colorectal Distension (Visceral Sensitivity)

Visceral Hypersensitivity Score was given on day 8 of administration to investigate the analgesic effects of HDCA and PULVIS FELLIS SUIS on the IBS mice model. The mice were anesthetized with 1% isoflurane, and transmitters were inserted into the abdominal cavity with electrodes tunneled through the abdominal wall. Colorectal distensions were measured through a distension catheter (volumes ranging from 30 μL to 90 μL, Fogarty catheter for arterial embolization, 4F) and evaluated as abdominal spasm. Electromyographic (EMG) responses were evaluated by dividing the area under the EMG curve (AUC) during colorectal distension by the EMG AUC during the 20-second limit recording before each distension, multiplied by 100, using the following equation:

$$AUC = ( AUC 20 s after distension / AUC 20 s before distension ) \times 100.$$

Data were calculated at distension pressures of 30, 60 and 90 μL.

### 3. In Vitro Study

Human QGP-1 cells were cultured in an RPMI-1640 medium supplemented with 10% fetal bovine serum (FBS), 100 U/mL penicillin and 100 g/mL streptomycin. Cells were incubated in a humidified atmosphere with 95% air and 5% CO₂ at 37° C. When the confluency reached 80%, the cells were seeded to a 96-well plate at a density of 5×10⁴ cells/well. After 24 h, the cells were incubated with different concentrations (10, 25, and 50 μM) of HCA species for 24 h, and the level of serotonin production in the culture medium was measured.

### 4. Sample Preparation

All tissue samples were kept in −80° C. fridges until use. For fecal and colon samples, a 10 mg sample was extracted by addition of 100 μL 95% methanol containing 125 ng/mL of L-tryptophan-d5, 50 ng/mL of cholic acid-d4, and 50 ng/mL of deoxycholic acid-d4. The samples were homogenized in TissueLyser II for 2 min and stored at 4° C. overnight. After centrifugation at 15,000 rpm for 5 min, the supernatant was collected for LC-MS/MS. A 10 μL serum sample was extracted by addition of 40 μL methanol containing internal standards, and stored overnight at −20° C. After centrifugation at 15,000 rpm for 5 min, the supernatant was collected for LC-MS/MS.

### 5. Metabolites Quantification

A targeted metabolomics analysis was performed using an Agilent 1290 Infinity II UPLC system coupled with a triple quadrupole (QQQ) 6470 mass spectrometer. An Agilent ECLIPSE PLUS C18 chromatographic column (2.1×5 mm, 1.8 μm) with a pre-column was used. For the LC-MS-QQQ analysis of serotonin, the mobile phase was solution A (water containing 0.1% formic acid) and solution B (acetonitrile containing 0.1% formic acid). The gradient was set as: 2% B (0-0.5 min), 2%-30% B (0.5-4 min), 30%-100% B (4-6 min), 100% B (6-8 min), 100%-2% B (8-8.5 min), and maintained at 2% B (8.5-10 min). For the LC-MS-QQQ analysis of HCA species, the mobile phase was solution A (water containing 0.1% formic acid) and solution B (acetonitrile containing 0.1% formic acid). The gradient was set as: 15% B (0-2 min), 15%-25% B (2-4 min), 25%-35% B (4-20 min), 35%-50% B (20-30 min), and 50%-100% B (30-33 min). Throughout the analysis, the column temperature was maintained at 40° C., the flow rate at 0.4 mL/min, and the sample injection volume at 2 μL. The TQ MS was operated in the positive/negative ion mode with a jet stream electrospray ionization (ESI) source. The ESI capillary voltage was set to 4,000 V and 3,500 V respectively, and the nozzle voltage to 1,000 V. The nitrogen drying gas had a flow rate of 10 L/min, and was heated to 350° C. The sheath gas was at a temperature of 350° C., and a flow rate of 8 L/min. The nebulizer pressure was 45 psi. The mass spectrometry was performed in multiple reaction monitoring (MRM) mode. MS data were collected and processed using Agilent software, and the data collected are shown in Table 1.

TABLE 1
MRM acquisition parameters for targeted metabolites

| | | | Prod | | | |
|---|---|---|---|---|---|---|
| No. | Name | Prec Ion | Ion | Frag (V) | CE (V) | Polarity |
| 1 | Tryptophan-d5 | 210.2 | 192.2 | 90 | 15 | Positive |
| | Tryptophan-d5 | 210.2 | 150 | 90 | 25 | Positive |
| 2 | Serotonin (5-HT) | 177.1 | 160 | 90 | 9 | Positive |
| | Serotonin (5-HT) | 177.1 | 115 | 90 | 29 | Positive |
| 3 | cholic acid-d4 (CA-d4) | 411 | 411 | 210 | 51 | Negative |
| 4 | Dehydrocholic acid-d4 (DCA-d4) | 395 | 349.3 | 250 | 35 | Negative |
| 5 | Hyocholic acid (HCA) | 407 | 407 | 250 | 10 | Negative |
| 6 | Hyodeoxycholic acid (HDCA) | 391 | 391 | 250 | 10 | Negative |
| 7 | Glycohyocholic acid (GHCA) | 464 | 74 | 250 | 39 | Negative |
| 8 | Glycohyodeoxycholic acid (GHDCA) | 448 | 74 | 250 | 35 | Negative |
| 9 | Taurohyocholic acid (THCA) | 514 | 80 | 250 | 87 | Negative |
| 10 | Taurohyodeoxycholic acid (THDCA) | 499 | 80 | 192 | 80 | Negative |

### 6. The Binding Affinity of HDCA to Recombinant Human TPH1

All experiments were performed on Biacore 8 k (GE Healthcare, Sweden). The recombinant human TPH1 was diluted to 40 g/mL using 10 mM acetate at pH 4.0 and covalently immobilized on parallel channels of a CM5 sensor chip surface for 10 min. The chip surface was activated with 0.4 M 1-(3-dimethylaminopropyl)-3-ethylcarbodiimide and 0.1 M N-hydroxysuccinimide at a flow rate of 10 μL/min for 7 min. The immobilized chip was blocked with 1 M ethanolamine at a flow rate of 10 μL/min for 7 min. HDCA was diluted in a running buffer containing phosphate buffered saline (PBS), 1 mM dithiothreitol, 0.5% Tween 20 and 5% dimethylsulfoxide (DMSO) to serial concentrations of 2.5, 5, 10, 25, 50, and 100 μM. HDCA was injected for 3 min at a flow rate of 30 μL/min for binding, and then dissociated for 1.5 min. All data were analyzed using kinetic models with Biacore 8 k Evaluation Software.

### 7. Statistics

Data were analyzed using GraphPad Prism 9.0, and the results are presented as mean±SEM. Statistical significance was assessed using t-test or one-way ANOVA, with p-values less than 0.05 regarded as statistically significant.

### Results

1) HCA Species were Significantly Reduced in Patients with IBS-D

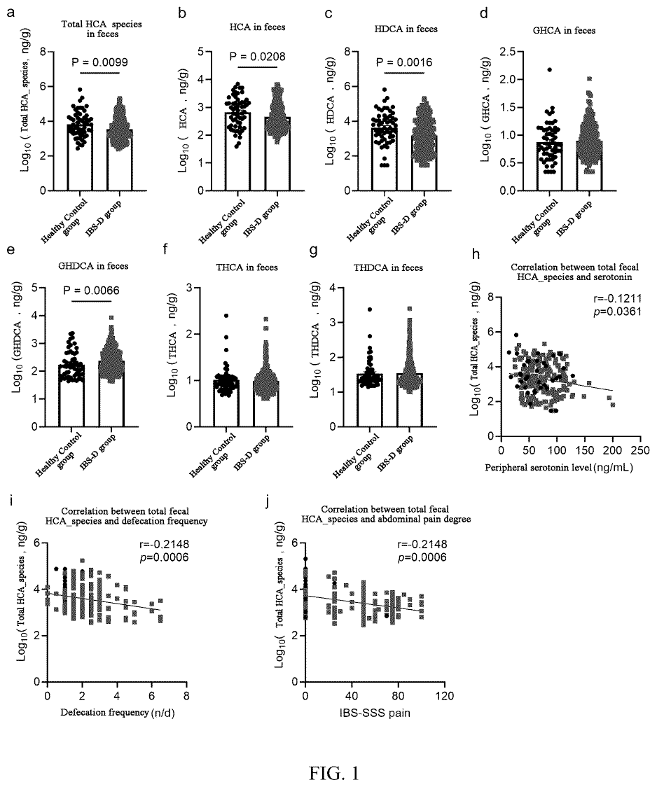

To identify gut-microbial metabolites that are associated with the serotonin level in IBS-D patients, a targeted metabolomics approach was used to search for metabolites that were significantly reduced in IBS-D patients and negatively correlated with the peripheral serotonin level. It was found that HCA species, including HCA, HDCA, GHCA, GHDCA, THCA, and THDCA, were significantly reduced in the feces of IBS-D patients, especially for HCA, HDCA and GHDCA (see FIG. 1 a-g). It was further found that the total HCA species were negatively correlated with the peripheral serotonin level in IBS-D patients (see FIG. 1 h), and also negatively correlated with the diarrhea and abdominal pain symptoms in IBS-D patients (see FIG. 1 i-j). These results indicate that HCA species may potentially improve the management of the diarrhea and abdominal pain symptoms of IBS-D and the regulation of abnormality in the peripheral serotonin level.

2) HCA Species were Regulated by Intestinal Microbiota

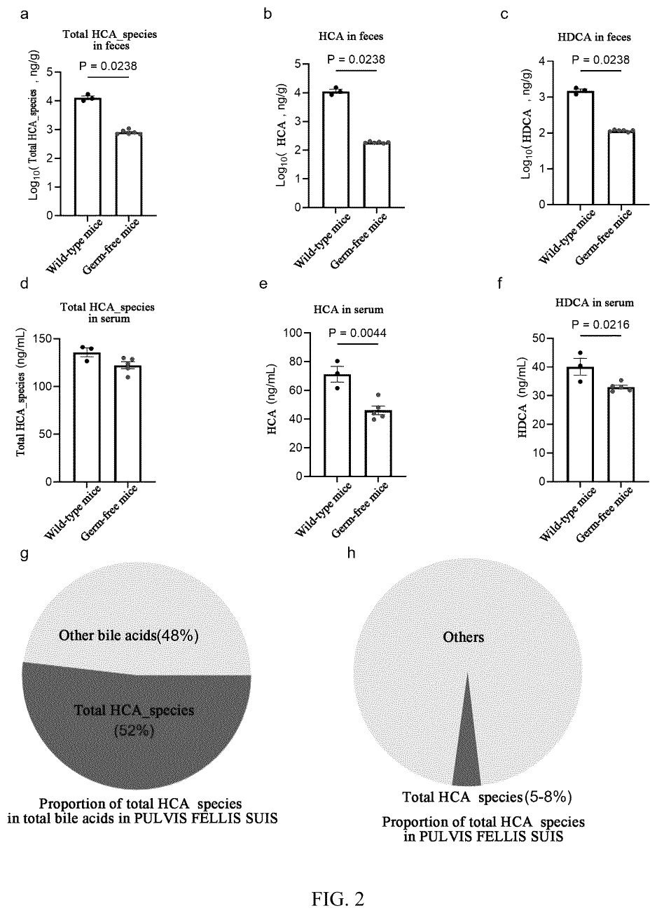

Next, the changes in HCA species in germ-free mice were measured, and it was found that the levels of HCA species, including HCA and HDCA, in the feces and serum of the germ-free mice were significantly reduced (see FIG. 2 a-f), indicating that HCA and HDCA are host-microbe co-metabolites that are significantly regulated by intestinal microbiota. However, HCA species constitute only a small proportion of the total bile acids in humans, approximately 2.43%, in contrast to the 76.34% in pigs. The inventors discovered that the traditional Chinese medicine PULVIS FELLIS SUIS contains a significant amount of HCA species. PULVIS FELLIS SUIS is the dried bile of Sus scrofa domesticus Brisson and is included in the Pharmacopoeia of the People's Republic of China (2020). This suggests that PULVIS FELLIS SUIS may serve as exogenous HCA-based bile acids supplemented to regulate the peripheral serotonin level for the treatment of gastrointestinal symptoms in patients with IBS-D (FIG. 2 g-h).

### 3) HDCA and PULVIS FELLIS SUIS Improved Diarrhea and Abdominal Pain Phenotypes in TNBS-Induced IBS Mice

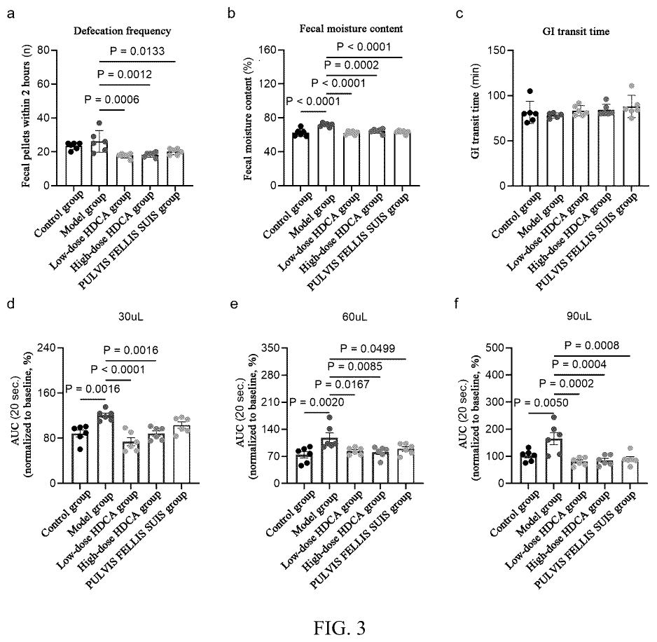

A mouse model of IBS induced with 2,4,6-trinitrobenzene sulfonic acid (TNBS) was used to evaluate the effects of HDCA and PULVIS FELLIS SUIS on diarrhea and abdominal pain. It was found that HDCA and PULVIS FELLIS SUIS significantly ameliorated the diarrhea and abdominal pain phenotypes in TNBS-induced IBS mice (n=6), such as GI transit, defecation frequency, fecal moisture content, and visceral hypersensitive abdominal electromyography (EMG) (see FIG. 3).

### 4) HCA Species Inhibited Serotonin Production In Vitro and In Vivo

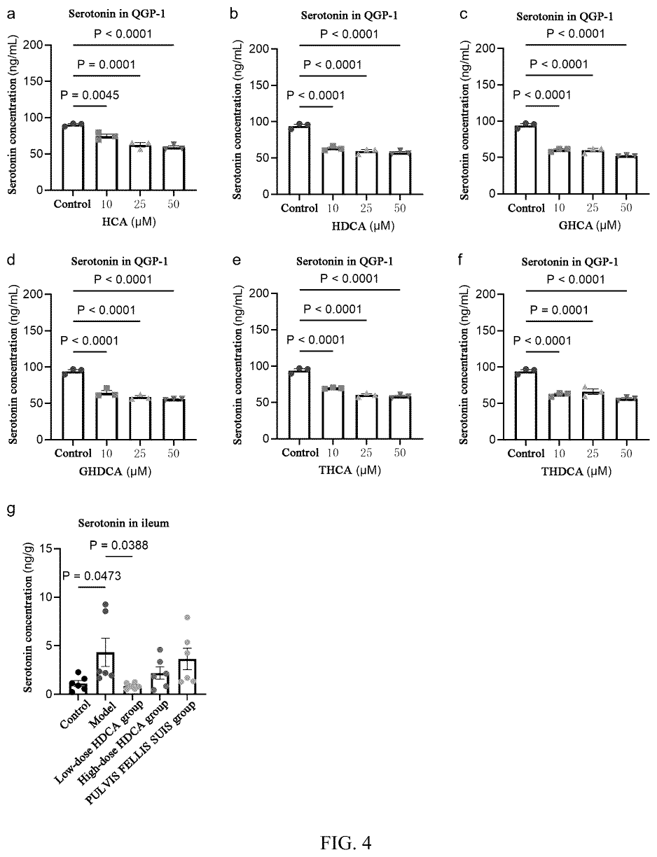

A QGP-1 cell line, a cell model capable of producing serotonin in vitro, was used to study the influence of HCA species on the serotonin synthesis by QGP-1 cells. As shown in FIG. 4, HCA species at a physiological and pharmacological concentration inhibited serotonin production. Moreover, both HDCA and PULVIS FELLIS SUIS suppressed the ileal serotonin level in TNBS-induced mice. These findings indicate that HCA species and PULVIS FELLIS SUIS can inhibit the peripheral serotonin level and improve the diarrhea and abdominal pain symptoms.

### 5) HDCA Suppressed Serotonin Biosynthesis by Inhibiting TPH1 Activity

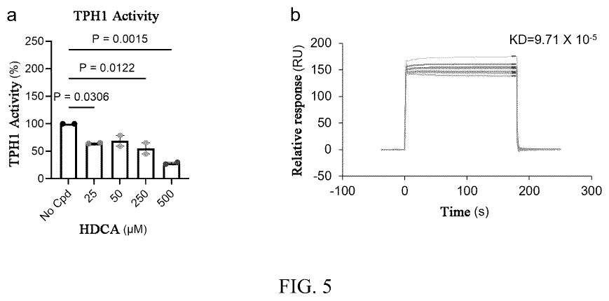

Based on the above findings, the present inventors hypothesized that HDCA may ameliorate the diarrhea and abdominal pain symptoms by regulating serotonin biosynthesis. Our findings showed that HDCA exerted an inhibitory effect on human TPH1 (see FIG. 5a). TPH1 is an enzyme responsible for catalyzing the conversion of tryptophan into 5-hydroxytryptophan, the precursor of serotonin. Subsequently, surface plasmon resonance (SPR) was used to determine the interaction between HDCA and recombinant human TPH1. It is noteworthy that HDCArapidly bound to the recombinant TPH1 with an equilibrium dissociation constant (K_{D}) of 9.71×10⁻⁵ M in a dose-dependent manner, indicating that HDCA directly targets TPH1 (see FIG. 5 b). This result indicates that HDCA is a potent inhibitor of TPH1, thereby regulating peripheral serotonin biosynthesis.

The above description of examples is intended to assist those skilled in the art in understanding and utilizing the invention. A person skilled in the art would certainly be able to make various modifications to these examples and apply the general principles disclosed herein to other examples without an inventive effort. Accordingly, the present invention is not limited to the above examples, and any modifications or variations made by those skilled in the art, without departing from the scope of the invention as disclosed, should fall within the scope of the invention.

What is claimed is:

1. A method for preventing or treating Irritable Bowel Syndrome with Diarrhea (IBS-D), comprising administering a therapeutically effective amount of a hyocholic acid based bile acid species (HCA species) and/or a crude drug comprising an HCA species to a subject in need thereof.

2. The method according to claim 1, wherein the HCA species comprises one or more compounds selected from the group consisting of hyocholic acid (HCA), hyodeoxycholic acid (HDCA), glycohyocholic acid (GHCA), glycohyodeoxycholic acid (GHDCA), taurohyocholic acid (THCA), and taurohyodeoxycholic acid (THDCA).

3. The method according to claim 1, wherein the crude drug comprising the HCA species is PULVIS FELLIS SUIS.

4. The method according to claim 1, wherein the HCA species or the crude drug comprising the HCA species is capable of inhibiting TPH1 activity or production of peripheral serotonin.

5. The method according to claim 1, wherein the HCA species and/or the crude drug comprising the HCA species is administered by injection, by administration via the respiratory tract, by administration via a cavity or tract, by mucosal administration, or by cutaneous administration.

6. The method according to claim 1, wherein the HCA species is administered in a dose of 0.1-50.0 mg/kg, and is administered at a frequency of once a day.

7. The method according to claim 1, wherein the HCA species or the crude drug comprising the HCA species is capable of improving diarrhea and abdominal pain phenotypes.

8. A pharmaceutical composition for treating IBS-D, comprising an HCA species and/or a crude drug comprising an HCA species.

9. The pharmaceutical composition according to claim 8, wherein a dosage form of the pharmaceutical composition includes dosage forms for injection, dosage forms for respiratory tract administration, dosage forms for administration via a cavity or tract, dosage forms for mucosal administration, or dosage forms for administration to or via skin.

10. A method for inhibiting TPH1 activity in a subject with IBS-D, comprising administering an HCA species or a crude drug comprising an HCA species to the subject.
